(19) United States
(12) Patent Application Publication
(10) Pub. No.: US 2018/0127687 A1
(43) Pub. Date: May 10, 2018

(54) DETERGENTS WITH IMPROVED DETERGENT POWER, CONTAINING AT LEAST ONE LACCASE

(71) Applicant: Henkel AG & Co. KGaA, Duesseldorf (DE)

(72) Inventors: Nina Mussmann, Willich (DE); Timothy O'Connell, Landsberg am Lech (DE); Daniela Herbst, Duesseldorf (DE); Inken Prueser, Duesseldorf (DE); Ralf G. Berger, Hannover (DE); Diana Linke, Rehburg (DE); Christoph Behrens, Hannover (DE)

(73) Assignee: Henkel AG & Co. KGaA, Duesseldorf (DE)

(21) Appl. No.: 15/569,765

(22) PCT Filed: Jun. 2, 2016
(86) PCT No.: PCT/EP2016/062440

§ 371 Date: Oct. 27, 2017

(30) Foreign Application Priority Data
Jun. 5, 2015 (DE) 10 2015 210 367.6
Jun. 5, 2015 (DE) 10 2015 210 368.4
Jun. 5, 2015 (DE) 10 2015 210 369.2
Jun. 5, 2015 (DE) 10 2015 210 370.6

(51) Int. Cl. C11D 3/386; C12N 9/02; C11D 11/00

(52) U.S. Cl. CPC C11D 3/38654; C11D 11/0017; C12N 9/0061

(57) ABSTRACT

A laccase is provided herein. The laccase includes an amino acid sequence which is at least about 70% identical to the amino acid sequence indicated in SEQ ID NO. 1, over the entire length thereof. A detergent is also provided herein. The detergent includes the at least one laccase. A method for washing textiles in aqueous solutions is also provided herein. The aqueous solutions include surfactants. The method includes utilizing a solution which includes surfactant and the at least one laccase.

### CROSS-REFERENCE TO RELATED APPLICATION

This application is a U.S. National-Stage entry under 35 U.S.C. § 371 based on International Application No. PCT/EP2016/062440, filed Jun. 2, 2016 which was published under PCT Article 21(2) and which claims priority to German Applications No. 102015210370.6, filed Jun. 5, 2015, 102015210369.2, filed Jun. 5, 2015, 102015210368.4, filed Jun. 5, 2015 and 102015210367.6, filed Jun. 5, 2015, which are all hereby incorporated in their entirety by reference.

### TECHNICAL FIELD

The present disclosure relates to the use of specific laccases during the washing of textiles, and to detergents containing said laccases.

### BACKGROUND

Laccases (EC 1.10.3.2) are copper-containing, “blue” enzymes which are present in many plants, fungi and microorganisms. Laccases rank among oxidoreductases. The catalytically active center contains four copper ions, which may differ in terms of their spectroscopic properties. The “blue” type 1 copper is involved in substrate oxidation; one type 2 and two type 3 copper ions form a trinuclear cluster which bonds oxygen and reduces to water. Laccases are also called p-diphenol oxidases. In addition to diphenols, laccases oxidize many other substrates, such as methoxy-substituted phenols and diamines. In respect of their substrates, laccases are surprisingly unspecific. Due to their broad substrate specificity and their ability to oxidize phenolic compounds, laccases have aroused great interest in industrial applications. Many promising areas for applying laccases include for example the delignification and the adhesion of fiberboards in the wood industry, coloring substances and decontaminating dye wastewaters in the textile industry, as well as use in biosensors.

With the help of mediators, i.e. intermediate molecules, laccases can also oxidize substrates which they would otherwise not be able to oxidize. The mediators are typically “small molecule compounds” which are oxidized by laccases. The oxidized mediator then in turn oxidizes the actual substrate.

The first laccase was found in 1883 in the Japanese lacquer tree (Rhus vernicifera). Laccases are found in many plants, such as peach, tomato, mango and potato; laccases are also known in some insects. However, the most frequently used laccases come from fungi, for example from the types agaricus, aspergillus, cerrena, curvularia, fusarium, lentinus, monocillium, myceliophthora, neurospora, penicillium, phanerochaete, phlebia, pleurotus, podospora, schizophyllum, sporotrichum, stagonospora and trametes.

In nature, the function of laccases consists inter alia in its involvement in decomposing lignocellulose, biosynthesis of cell walls, browning of fruit and vegetables, as well as preventing microbial attacks on plants.

It is often difficult to remove colored stains/impurities effectively from dirty laundry or from a dirty object. Removing strong-colored stains and impurities, i.e. those coming from fruit and/or vegetables, is particularly demanding. These stains and impurities contain colored pigments based on carotenoid compounds, such as a-, b- and g-carotene and lycopene, porphyrins, such as chlorophyll, and flavonoid pigments and dye components. This last group of dye components based on natural flavonoid comprises the strongly-colored anthocyanin dyes and pigments based on pelargonidin, cyanidin, delphinidin and the methyl esters and anthoxanthins thereof. These compounds are the origin of most orange-colored, red, violet and blue colors in fruits and are abundant in all berries, cherries, redcurrants and blackcurrants, grapefruits, passion fruit, oranges, lemons, apples, pears, pomegranates, red cabbage, beetroots and also in flowers. Derivatives of cyanidin are present in up to 80% of pigmented leaves, in up to 70% of fruits and in up to 50% of flowers. Special examples of such impurities include impurities from tea, coffee, spices such as curry and paprika, orange, tomato, banana, tea, mango, broccoli, carrot, beetroot, spinach and grass. Ballpoint pen ink is also known to be a stain which is very difficult to remove. These strongly colored flavonoid and carotenoid dyes are often polycyclic and heterocyclic compounds with systems of a conjugated double bond. This chemical structure is often responsible for the clear color of the compounds.

The named difficulties in removing colored stains and impurities are particularly severe when formulating liquid detergents and cleaning agents, as these do not usually contain any bleaching agents.

### BRIEF SUMMARY

A laccase is provided herein. The laccase includes an amino acid sequence which is at least about 70% identical to the amino acid sequence indicated in SEQ ID NO. 1, over the entire length thereof.

A detergent is also provided herein. The detergent includes the at least one laccase.

A method for washing textiles in aqueous solutions is also provided herein. The aqueous solutions include surfactants. The method includes utilizing a solution which includes surfactant and the at least one laccase.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has surprisingly been found that a laccase of SEQ. ID NO.1 thus far unknown from the Pleurotus pulmonarius, or laccases sufficiently similar thereto (relative to the sequence identity), are particularly suitable for use in liquid detergents or cleaning agents, and are improved with respect to detergent power.

A first subject matter of the present disclosure is therefore a laccase which comprises an amino acid sequence which is at least about 70%, and increasingly preferably at least about 75%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5% and about 99% identical to the amino acid sequence indicated in SEQ ID NO. 1, over the entire length thereof.

A further subject matter of the present disclosure is a laccase which is coded by a nucleic acid sequence which is at least about 70%, and increasingly preferably at least about 75%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5% and about 99% identical to the nucleic acid sequence indicated in SEQ ID NO. 2, over the entire length thereof.

A further subject matter of the present disclosure is a detergent which contains at least one laccase as contemplated herein, i.e. a laccase which comprises an amino acid sequence which is at least about 70% and increasingly preferably at least about 75%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5% and about 99% identical to the amino acid sequence indicated in SEQ ID NO. 1, over the entire length thereof, or a laccase which is coded by a nucleic acid sequence which is at least about 70% and increasingly preferably at least about 75%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5% and about 99% identical to the nucleic acid sequence indicated in SEQ ID NO. 2, over the entire length thereof.

Advantageously, the agent as contemplated herein has an improved detergent power, in particular when removing colored stains and impurities. Particularly preferably, the agent as contemplated herein is suitable for removing anthocyanin-containing impurities.

SEQ ID NO. 1 is the sequence of a laccase from the basidiomycetes Pleurotus pulmonarius; DSMZ 5331 (DSMZ: Deutsche Sammlung von Mikroorganismen and Zellkulturen).

SEQ ID NO. 2 is the cDNA coding for the laccase with the SEQ ID NO. 1, i.e. without introns.

Furthermore, it has surprisingly been found that also the following laccases are particularly suitable for use in liquid detergents or cleaning agents and are improved with regard to detergent power:

a) a laccase [SEQ. ID NO.3] from the basidiomycetes Pleurotus ostreatus var. florida or laccases sufficiently similar hereto (relative to the sequence identity);
b) a laccase unknown thus far [SEQ. ID NO.5] from the Shiitake fungus (Lentinula edodes) or laccases sufficiently similar hereto (relative to the sequence identity);
c) a laccase unknown thus far [SEQ. ID NO.7] from Trametes species (Tsp, DSMZ 11309) (DSMZ: Deutsche Sammlung von Mikroorganismen and Zellkulturen), or laccases sufficiently similar hereto (relative to the sequence identity).

A further subject matter of the present disclosure is therefore a laccase which comprises an amino acid sequence which is at least about 70%, and increasingly preferably at least 75%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% and 99% identical to the amino acid sequence indicated in SEQ ID NO. 3, SEQ ID NO.5, and/or SEQ ID NO.7, over the entire length thereof.

Further subjects of the present disclosure are therefore detergents which contain at least one laccase which comprises an amino acid sequence which is at least 70%, and increasingly preferably at least about 75%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5% and about 99% identical to the amino acid sequence indicated in SEQ ID NO. 3, SEQ ID NO.5, and/or SEQ ID NO.7, over the entire length thereof.

Advantageously, the agent as contemplated herein has an improved detergent power, in particular when removing colored stains and impurities. Particularly preferably, the agent as contemplated herein is suitable for removing anthocyanin-containing impurities.

SEQ ID NO. 3 is the sequence of a laccase from Pleurotus ostreatus var. florida from the publication Palmieri, G. et al. (2003) Atypical laccaseisoenzymes from coppersupplemented Pleurotus ostreatus cultures. EnzymeMicrob. Technol. 33(2-3), 220-230. The sequence is on page 227 of the publication.

SEQ ID NO. 5 is the sequence of a laccase from the basidiomycetes Lentinula edodes (Shitake).

SEQ ID NO. 7 is the sequence of a laccase from Trametes species (Tsp, DSMZ 11309) (DSMZ: Deutsche Sammlung von Mikroorganismen and Zellkulturen).

A further subject matter of the present disclosure is a detergent which contains at least one laccase which is coded by a nucleic acid sequence which is at least 70%, and increasingly preferably at least about 75%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5% and about 99% identical to the nucleic acid sequence indicated in in SEQ ID NO. 4, SEQ ID NO. 6 or SEQ ID NO. 8, over the entire length thereof.

SEQ ID NO. 4 is the cDNA coding for the laccase with the SEQ ID NO. 3, i.e. without introns.

SEQ ID NO. 6 is the cDNA coding for the laccase with the SEQ ID NO. 5, i.e. without introns.

SEQ ID NO. 8 is the cDNA coding for the laccase with the SEQ ID NO. 7, i.e. without introns.

The identity of nucleic acid or amino acid sequences is determined by a sequence comparison. This sequence comparison is based on the BLAST algorithm established in the prior art and conventionally used (cf. for example Altschul, S. F., Gish, W., Miller, W., Myers, E. W. & Lipman, D J. (1990) “Basic local alignment search tool.” J. Mol. Biol. 215:403410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, pp. 3389-3402) and takes the form of similar sequences of nucleotides or amino acids being assigned to one another in the nucleic acid sequence or amino acid sequence. A tabulated allocation of the respective positions is called an alignment. A further algorithm available in the prior art is the FASTA algorithm. Sequence comparisons (alignments), in particular multiple sequence comparisons, are produced with computer programs. There are frequently used, for example, the Clustal series (cf. for example Chenna et al. (2003): Multiple sequence alignment with the Clustal series of programs. Nucleic Acid Research 31, 3497-3500), T-Coffee (cf. for example Notredame et al. (2000): T-Coffee: A novel method for multiple sequence alignments. J. Mol. Biol. 302, 205-217) or programs which are based on these programs or algorithms. In the present patent application, all sequence comparisons (alignments) were produced using the computer program Vector NTI® Suite 10.3 (Invitrogen Corporation, 1600 Faraday Avenue, Carlsbad, Calif., USA) with the given standard parameters, the AlignX module of which for the sequence comparisons is based on ClustalW.

Such a comparison also makes it possible to comment on the similarity of compared sequences. It is usually indicated in percentage of identity, i.e. the proportion of identical nucleotides or amino acid residues at the same positions or in an alignment corresponding to another position. In amino acid sequences, the more broadly formulated term of homology relates to conserved amino acid exchange, thus amino acids with similar chemical activity, as these exert mostly similar chemical activities within the protein. Therefore, the similarity between compared sequences can also be indicated as the percentage of homology or percentage of similarity. Details of identity and/or homology can be found via whole polypeptides or genes or only via individual regions. Homologous or identical regions of different nucleic acid sequences or amino acid sequences are therefore defined by similarities in the sequences. Such regions often have identical functions. They can be small and comprise only a few nucleotides or amino acids. Often, such small regions carry out essential functions for the overall activity of the protein. Therefore, it may be expedient to relate sequence similarities only to individual, possibly small regions. However, unless otherwise indicated, in the present disclosure identity or homology details relate to the overall length of the respectively indicated nucleic acid sequence or amino acid sequence.

The laccases as contemplated herein and the laccases which can be used in the detergents as contemplated herein can be obtained from plants, fungi and preferably bacteria, in particular from bacilli and actinomycetes. The laccase comprising SEQ ID NO. 1 can be obtained from Pleurotus pulmonarius. The laccase comprising SEQ ID NO.3 can be obtained from Pleurotus ostreatus var. florida. The laccase comprising SEQ ID NO.5 can be obtained from Lentinula edodes. The laccase comprising SEQ ID NO.7 can be obtained from Trametes species.

However, the natural production quantities of laccases are often very low. Therefore, it may be expedient to increase production by having laccase genes expressed in foreign production hosts.

For this purpose, generally vectors are used which contain a nucleic acid which codes for a laccase as contemplated herein.

These can be DNA molecules or RNA molecules. They can be present as a single strand, as a single strand complementary to this single strand or as a double strand. In particular, with DNA molecules, the sequences of two complementary strands are to be considered in respectively all three possible reading frames. Furthermore, it is to be considered that different codons, i.e. base triplets, can code for the same amino acids, with the result that a specific amino acid sequence can be coded from several different nucleic acids. A person skilled in the art is capable of determining these nucleic acid sequences unequivocally, as, in spite of the genetic code having degenerated, individual codons can be assigned to defined amino acids. Therefore, proceeding from an amino acid sequence, a person skilled in the art can ascertain nucleic acids coding for this amino acid sequence without problems. Furthermore, in nucleic acids one or more codons can be replaced by synonymous codons. This aspect relates in particular to the heterologous expression of the enzymes as contemplated herein. In this way, every organism, for example a host cell of a production strain, possesses a specific codon use. Codon use is understood to mean the translation of genetic code into amino acids by the respective organism. There can be shortages in the protein biosynthesis if the codons on the nucleic acid are faced with a comparably low number of charged tRNA molecules in the organism. Although coding for the same amino acid, this leads to a codon being translated less efficiently in the organism than a synonymous codon which codes for the same amino acid. This can be translated more efficiently in the organism because of the presence of a higher number of tRNA molecules for the synonymous codon.

Using methods known generally nowadays to a person skilled in the art, such as for example the chemical synthesis or the polymerase chain reaction (PCR) in conjunction with molecular-biological and/or protein-chemical standard methods, it is possible to produce the corresponding nucleic acids up to the complete genes, using known DNA and/or amino acid sequences. Such methods are known for example from Sambrook, J., Fritsch, E. F. and Maniatis, T. 2001. Molecular cloning: a laboratory manual, 3rd Edition Cold Spring Laboratory Press.

Within the meaning of the present disclosure, vectors are understood to mean elements including of nucleic acids, which elements contain a nucleic acid coding for a laccase as contemplated herein as a characterizing nucleic acid region. They are capable of establishing these as a stable genetic element, in a species or a cell line, over several generations or cell divisions. Vectors are special plasmids, thus circular genetic elements, in particular when in use in bacteria. Within the framework of the present disclosure, a nucleic acid coding for a laccase as contemplated herein is cloned in a vector. The vectors include for example those whose origins are of bacterial plasmid, viruses or bacteriophages, or predominantly synthetic vectors or plasmids with elements of the most varied origin. With the other genetic elements respectively available, vectors are capable of establishing themselves as stable units in the relevant host cells over several generations. They can be present extra-chromosomally as separate units or be integrated into a chromosome or chromosomal DNA.

Expression vectors comprise nucleic acid sequences which enable it to replicate preferably microorganisms, particularly preferably bacteria, in the hosts cells containing them, and to express a nucleic acid contained therein. The expression is influence in particular by the promoter(s) which regulate transcription. In principle, the expression can take place by the natural promoter located originally before the nucleic acid to be expressed, but also by a promoter of the host cell provided on the expression vector or also by a modified or completely different promoter of another organism or another host cell. In the present case, at least one promoter is made available for the expression of a nucleic acid coding for a laccase as contemplated herein, and used for the expression thereof. It may also be possible to regulate expression vectors, for example by changing the cultivation conditions or upon reaching a specific cell density of the host cells containing same, or by adding specific substances, in particular activators of gene expression. An example of such a substance is the galactose derivative isopropyl β-D-1-thiogalactopyranoside (IPTG) which is used as an activator of the bacterial lactose operons (lac-operons). In contract with expression vectors, the contained nucleic acid is not expressed in cloning vectors.

Preferably, a nucleic acid coding for a laccase as contemplated herein or a vector containing such a nucleic acid is transformed into a microorganism which then serves as host cell. Alternatively, individual components, i.e. nucleic acid parts or fragments of a nucleic acid coding for a laccase as contemplated herein can be introduced into a host cell such that the resulting host cell contains one such nucleic acid or one such vector. This process is particularly suitable if the host cell already contains one or more components or several components of such a nucleic acid or of such a vector and the further components are then correspondingly supplemented. Methods for transforming cells are established in the prior art and are sufficiently known to a person skilled in the art. In principle, all cells, i.e. procaryotic or eucaryotic cells, are suitable as host cells. Those host cells are preferred which can advantageously be genetically manipulated, which for example affects the transformation with the nucleic acid or the vector and the stable establishing thereof, for example single-celled fungi or bacteria. Furthermore, preferred host cells are exemplified by a good microbiological and biotechnological handling behavior. For example, this relates to easy cultivability, high growth rates, small demands on fermentation media and good production and secretion rates for foreign proteins. Preferred host cells as contemplated herein secrete the (transgenically) expressed protein into the medium surrounding the host cells. Furthermore, the laccases can be modified after production by the cells producing same, for example by linking sugar molecules, formylations, aminations etc. Such post-translational modifications can functionally influence the laccases.

Such host cells which have activity which can be regulated because of genetic regulation elements, which are for example available at the vector, but may also be present in advance in these cells, are particularly suitable for producing laccases as contemplated herein. For example, by monitored addition of chemical compounds which serve as activators, by changing the cultivation conditions or when achieving a specific cell density, these can be encouraged to express. This makes possible an economic production of the proteins as contemplated herein. An example of such a connection is the IPTG, as described previously.

Preferred host cells are procaryotic or bacterial cells. Bacteria are exemplified by short generation times and small demands on the cultivation conditions. As a result, cost-favorable cultivation methods or production methods are established. Additionally, a person skilled in the art of fermentation technology will have substantial experience with bacteria. From the most varied reasons, such as sources of nutrients, product formation rate, time needed, etc., to be established experimentally in the individual case, gram-negative or gram-positive bacteria may be suitable for special production.

With gram-negative bacteria, such as, for example, Escherichia coli, a plurality of proteins are secreted in the periplasmatic space, thus into the compartment between the membranes enclosing the cells. This can be advantageous for special applications. Furthermore, gram-negative bacteria can also be developed such that they expel the expressed proteins not only in the periplasmatic space, but in the medium surrounding the bacterium. Gram-positive bacteria such as for example bacilli or actinomycetes or other representatives of the actinomycetales have, on the other hand, no outer membrane, with the result that secreted proteins are emitted immediately in the medium surrounding the bacteria, generally the nutrient medium, from which the expressed proteins can be purified. They can be isolated directly from the medium or further processed. Furthermore, gram-positive bacteria are related or identical to most original organisms for technically important enzymes, and themselves form mostly comparable enzymes, with the result that they have a similar codon use and the protein synthesis apparatus thereof is naturally correspondingly aligned.

The named host cells can be changed with regard to their demands on culture conditions, or have different or additional selection markers, or express different or additional proteins. In particular, these can also be those host cells which express several proteins or enzymes transgenically.

The present disclosure can in principle be applied to all microorganisms, in particular to all fermentable microorganisms, and leads to laccases as contemplated herein being able to be produced by the use of such microorganisms.

Particularly preferred host cells for obtaining the laccases as contemplated herein are bacteria, in particular those which are selected from the genera Escherichia, Klebsiella, Bacillus, Staphylococcus, Corynebacterium, Arthrobacter, Streptomyces, Stenotrophomonas and Pseudomonas and in particular from Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor and Stenotrophomonas maltophilia.

However, the host cell can also be a eucaryotic cell, which exemplifiedhas a cell nucleus. In contrast to procaryotic cells, eucaryotic cells can modify the formed protein in post-translational manner. Examples of these are fungi such as actinomycetes or yeasts such as Saccharomyces or Kluyveromyces. This can then for example be particularly advantageous if, in conjunction with their synthesis, the proteins are intended to experience specific modifications which such systems make possible. The modifications which carry out eucaryotic systems particularly in conjunction with the protein synthesis include for example bonding low-molecular compounds such as membrane anchors or oligosaccharides. Such oligosaccharide modifications may be desirable for example for reducing the allergenicity of an expressed protein. Also, a coexpression with the enzymes formed naturally by such cells, such as for example cellulases or lipases, may be advantageous. Furthermore, for example thermophilic fungal expression systems may be suitable particularly for expressing temperature-resistant proteins or variants.

The named host cells are cultivated and fermented conventionally, for example in discontinuous or continuous systems. In the first case, a suitable nutrient medium is inoculated with the host cells and the product is harvested from the medium after a period of time to be determined experimentally. Continuous fermentations are exemplified by achieving a dynamic equilibrium in which cells partly die off over a comparably long period of time, but also grow back and the formed protein can be removed from the medium simultaneously.

Fermentation methods are known per se from the prior art and represent the actual industrial production step, generally followed by a suitable purification method of the produced product, for example of a laccase as contemplated herein.

Fermentation methods which are exemplified in that the fermentation is carried out via a feed strategy are in particular considered. The media components which are consumed by the continuous cultivation are supplemented by this. As a result, considerable increases can be achieved in cell density and also in cell mass or dry mass and/or in particular in the activity of the laccase of interest. Furthermore, the fermentation can also be developed such that undesired metabolic products are filtered out or neutralized by adding buffer or respectively suitable counterions.

The produced laccase can be harvested from the fermentation medium. Such a fermentation method is preferred vis-à-vis an isolation of the laccase from the host cell, i.e. a product preparation from the cell mass (dry mass), but requires the provision of suitable host cells or of one or more suitable secretion markers or mechanisms and/or transport systems, in order that the host cells secrete the laccase into the fermentation medium. Without secretion, alternatively the isolation of the laccase from the host cell, i.e. a purification of the same from the cell mass, can take place, for example using conventional methods of enzyme chemistry such as salt precipitation, ultrafiltration, ion-exchange chromatography and hydrophobic interaction chromatography. The purification can be monitored by SDS polyacrylamide gel electrophoreses. The enzyme activity of the purified enzyme at different temperatures and pH values can be determined; similarly, the molecular weight and the isoelectric point can be determined.

Surprisingly, it has been found that the laccases as contemplated herein, in particular the laccase comprising SEQ ID NO. 1 and also the laccase comprising SEQ ID NO.3, SEQ ID NO.5 and SEQ ID NO.7 improve the detergent power in particular of liquid detergent formulations and do not, as known from other laccases, bring about the undesired darkening and thus intensification of stains, in particular tea and coffee stains.

The concentration of laccases in the detergent as contemplated herein is of from about 0.001 to about 0.15 wt.-%, preferably of from about 0.005 to about 0.06 wt.-%, relative to active protein.

The detergent as contemplated herein can be used preferably in the temperature range of from about 5° C. to about 95° C., preferably from about 20° C. to about 60° C. and particularly preferably from about 30° C. to about 40° C.

The detergent as contemplated herein can contain additional mediators, in order to oxidize the dyes to be removed with higher efficiency. Mediators suitable as contemplated herein are for example Tempo (2,2,6,6-tetramethyl-1-piperidinyloxy), HBT (1-hydroxybenzotriazol), ABTS (2,2′-azinobis-3-ethylbenzothiazole-6-sulfonate), NHA (N-hydroxy-acetanilide), 2,5-xylidine, ethanol, copper, 4-methylcatechol, N-hydroxyphthalimide, gallic acid, tannic acid, quercetin, syringic acid, guaiacol, dimethoxybenzyl alcohol, phenol, violuric acid (isonitrosobarbituric acid), phenol red, bromophenol blue, cellulose, p-coumarin acid, Rooibos, o-cresol, dichloroindophenol, hydroxybenzotriazol, cycloheximide or vanillin.

A further subject matter of the present disclosure is the use of a laccase as contemplated herein for improving the detergent power of a detergent, in particular when removing colored stains and impurities, particularly preferably when removing impurities containing anthocyanin.

A further subject matter of the present disclosure is a method for washing textiles in aqueous solutions containing surfactant, which is exemplified in that an aqueous solution containing surfactant is used which contains at least one laccase as contemplated herein.

The method is achieved in its simplest form in that textiles requiring cleaning are brought into contact with the aqueous liquor, wherein a conventional washing machine can be used or the laundry can be carried out by hand. It is preferred, as contemplated herein, to carry out the method accompanied by intensive ventilation of the aqueous liquor as is the case when using a conventional domestic washing machine program.

In addition to the laccases suitable for removing the named strains and impurities, a detergent can contain ingredients which are normally compatible with this component. Thus, for example, it can additionally still contain one or more color-transfer inhibitors, these then preferably in quantities of from about 0.1 wt.-% to about 2 wt.-%, in particular from about 0.2 wt.-% to about 1 wt.-%, which are selected in a preferred embodiment from the polymers of vinylpyrrolidone, vinylimidazole, vinylpyridin-N-oxide or the copolymers of same. Both polyvinylpyrrolidones with molecular weights of from about 15,000 g/mol to about 50,000 g/mol and also polyvinylpyrrolidones with higher molecular weight of for example up to more than 1,000,000 g/mol, in particular of from about 1,500,000 g/mol about to 4,000,000 g/mol, N-vinylimidazole/N-vinylpyrrolidone copolymers, polyvinyloxazolidone, copolymers based on vinyl monomers and carboxylic acid amides, polyesters and polyamides containing pyrrolidone groups, grafted polyamidoamines and polyethylene imines, polyamine-N-oxide polymer and polyvinyl alcohols can be used. Enzymatic systems comprising a peroxidase and hydrogen peroxide or a substance providing hydrogen peroxide in water can also be used. The addition of a mediator compound for the peroxidase, for example an acetosyringone, a phenol derivative or a phenothiazine or phenoxazine, is preferred in this case, wherein also additionally, the above-mentioned polymeric color-transfer inhibitor active ingredients can be used. Polyvinylpyrrolidone preferably has an average molar mass in the range of from about 10,000 g/mol to about 60,000 g/mol, in particular in the range of from about 25,000 g/mol to about 50,000 g/mol. Among copolymers, those made of vinylpyrrolidone and vinylimidazole in the molar ratio from about 5:1 to about 1:1 with an average molar mass in the range of from about 5,000 g/mol to about 50,000 g/mol, in particular from about 10,000 g/mol to about 20,000 g/mol, are preferred.

Detergents which can be present as in particular powdery solids, in redensified particle form, in granular form, as homogeneous solutions or suspensions, can in principle also contain all ingredients known and commonplace in such agents, apart from the laccases used as contemplated herein. The agents as contemplated herein can contain in particular builders, surface-active surfactants, bleaching agents on the basis of organic and/or inorganic peroxygen compounds, bleach activators, water-miscible organic solvents, enzymes, sequester agents, electrolytes, pH regulators and further auxiliaries, such as optical brighteners, graying inhibitors, foam regulators as well as dyes and fragrances.

The agents contain preferably a surfactant or several surfactants, wherein in particular anionic surfactants, non-ionic surfactants and mixtures thereof, but also cationic, zwitterionic and amphoteric surfactants come into consideration.

Suitable non-ionic surfactants are in particular alkyl glycosides and ethoxylation and/or propoxylation products of alkyl glycosides or linear or branched alcohols each with 12 to 18 C atoms in the alkyl portion and 3 to 20, preferably 4 to 10 alkyl ether groups. Furthermore, corresponding ethoxylation and/or propoxylation products of N-alkylamines, vicinal diols, fatty acid esters and fatty acid amides which, with regard to the alkyl portion, correspond to the named long-chain alcohol derivatives, as well as alkyl phenols with 5 to 12 C atoms in the alkyl residue, can be used.

Preferably alkoxylated, advantageously ethoxylated, in particular primary alcohols with preferably 8 to 18 C atoms and on average 1 to 12 mols ethylene oxide (EO) per mol alcohol are used as non-ionic surfactants, in which the alcohol residue can be linear or preferably methyl-branched in 2 position or can contain linear and methyl-branched residues in the mixture, as are conventionally present in oxo alcohol residues. However, in particular alcohol ethoxylates with linear residues made of alcohols of native origin with 12 to 18 C atoms, e.g. made of coco, palm, tallow fat or oleyl alcohol, and on average 2 to 8 EO per mol alcohol, are preferred. The preferred ethoxylated alcohols include for example C₁₂-C₁₄ alcohols with 3 EO or 4 EO, C₉-C₁₁ alcohols with 7 EO, C₁₃-C₁₅ alcohols with 3 EO, 5 EO, 7 EO or 8 EO, C₁₂-C₁₈ alcohols with 3 EO, 5 EO or 7 EO and mixtures thereof, such as mixtures of C₁₂-C₁₄ alcohol with 3 EO and C₁₂-C₁₈ alcohol with 7 EO. The indicated degrees of ethoxylation represent statistical averages which can be an integer or a fractional number for a special product. Preferred alcohol ethoxylates have a concentrated homolog distribution (narrow range ethoxylates, NRE). In addition to these non-ionic surfactants, fatty alcohols with more than 12 EO can thus be used. Examples of this are (tallow) fatty alcohols with 14 EO, 16 EO, 20 EO, 25 EO, 30 EO or 40 EO. Conventional extremely low-foaming compounds are used, in particular in agents for use in machine methods. These include preferably C₁₂-C₁₈ alkyl polyethylene glycol polypropylene glycol ether, each with up to 8 mol ethylene oxide and propylene oxide units in the molecule. However, other known low-foaming non-ionic surfactants can also be used, such as for example C₁₂-C₁₈ alkyl polyethylene glycol polybutylene glycol ether each with up to 8 mol ethylene oxide and butylene oxide units in the molecule as well as end-group sealed alkyl polyalkylene glycol mixed ether. The hydroxyl-group containing alkoxylated alcohols, so-called hydroxy mixed ethers, are also particularly preferred. The non-ionic surfactants which can be used also include alkyl glycosides of the general formula RO(G)ₓ, in which R means a primary straight-chained or methyl-branched aliphatic residue, in particular methyl-branched in 2 position, with 8 to 22, preferably 12 to 18 C atoms and G stands for a glucose unit with 5 or 6 C atoms, preferably for glucose. The degree of oligomerization x, which indicates the distribution of monoglycosides and oligoglycosides, is any number—which can also take on fractional values as variables to be determined analytically—between about 1 and about 10; preferably x is from about 1.2 to about 1.4. Also suitable are polyhydroxy fatty acid amides of the formula

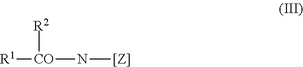

in which RICO stands for an aliphatic acyl residue with 6 to 22 carbon atoms, R² for hydrogen, an alkyl or hydroxyalkyl residue with 1 to 4 carbon atoms and [Z] for a linear or branched polyhydroxyalkyl residue with 3 to 10 carbon atoms and 3 to 10 hydroxyl groups.

Preferably, the polyhydroxy fatty acid amides are derived from reducing sugars with 5 or 6 carbon atoms, in particular from glucose. The group of polyhydroxy fatty acid amides also includes compounds of the formula

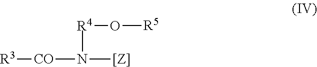

in which R³ stands for a linear or branched alkyl or alkenyl residue with 7 to 12 carbon atoms, R⁴ for a linear, branched or cyclic alkylene residue or an arylene residue with 2 to 8 carbon atoms and R⁵ for a linear, branched or cyclic alkyl residue or an aryl residue or an oxyalkyl residue with 1 to 8 carbon atoms, wherein C₁-C₄ alkyl or phenyl residues are preferred, and [Z] for a linear polyhydroxyalkyl residue, the alkyl chain of which is substituted with at least two hydroxyl groups, or alkoxylated, preferably ethoxylated or propoxylated derivatives of this residue. [Z] is also preferably obtained here by reductive amination of a sugar such as glucose, fructose, maltose, lactose, galactose, mannose or xylose. The N-alkoxy-substituted or N-aryloxy-substituted compounds can be converted into the desired polyhydroxy fatty acid amides by reaction with fatty acid methyl esters in the presence of an alkoxide as a catalyst. A further class of preferably used non-ionic surfactants which are used either as a single non-ionic surfactant or in combination with other non-ionic surfactants, in particular together with alkoxylated fatty alcohols and/or alkyl glycosides, are alkoxylated, preferably ethoxylated or ethoxylated and propoxylated fatty acid alkyl esters, preferably with 1 to 4 carbon atoms in the alkyl chain, in particular fatty acid methyl ester. Also, non-ionic surfactants of the type of amine oxides, for example N-coco-alkyl-N,N-dimethylamine oxide and N-tallow alkyl-N,N-dihydroxyethyl amine oxide, and the fatty acid alkanolamides can be suitable. The quantity of these non-ionic surfactants is preferably not more than that of the ethoxylated fatty alcohols, in particular not more than half thereof. So-called Gemini surfactants come into consideration as further surfactants. Generally, these mean those compounds which have two hydrophilic groups per molecule. These groups are generally separated from one another by a so-called “spacer”. This spacer is generally a carbon chain which should be long enough for the hydrophilic groups to have a sufficient distance so that they can act independently of one another. Such surfactants are generally exemplified by an unusually small critical micelle concentration and the capability of strongly reducing the surface tension of the water. In exceptional cases, the expression Gemini surfactants is understood to mean not only such “dimeric”, but also corresponding “trimeric” surfactants. Suitable Gemini surfactants are for example sulfated hydroxy mixed ether or dimer alcohol-bis- and trimer alcohol-tris-sulfates and -ether sulfates. End-group sealed dimeric and trimeric mixed ethers are exemplified in particular by their bifunctionality and multifunctionality. Thus, the named end-group sealed surfactants have good cross-linking properties and are foam-poor, with the result that they are suitable in particular for use in machine washing or cleaning methods. However, Gemini polyhydroxy fatty acid amides or poly-polyhydroxy fatty acid amides can also be used.

Suitable anionic surfactants are in particular soaps and those which contain sulfate or sulfonate groups. As sulfonate-type surfactants preferably C₉-C₁₃ alkylbenzene sulfonates, olefin sulfonates, i.e. mixtures of alkene and hydroxy alkane sulfonates and disulfonates, as for example are obtained C₁₂-C₁₈ monoolefins with terminal or internal double bond by sulfonating with gaseous sulfur trioxide and then alkali or acid hydrolysis of sulfonation products, come into consideration. Also, alkane sulfonates which are obtained from C₁₂-C₁₈ alkanes for example by sulfo chlorination or sulfoxidation with subsequent hydrolysis or neutralization are suitable. Also, the esters of α-sulfofatty acids (estersulfonates) are suitable, for example the α-sulfonated methyl esters of hydrogenated coco fatty acids, palm kernel fatty acids or tallow fatty acids, which are produced by α-sulfonation of methyl esters of fatty acids of plant and/or animal origin with 8 to 20 C atoms in the fatty acid molecule and subsequent neutralization to water-soluble mono salts. Preferably here it is the α-sulfonated esters of the hydrogenated coco fatty acids, palm kernel fatty acids or tallow fatty acids, wherein also sulfonation products of unsaturated fatty acids, for example oleic acid, may be present in small quantities, preferably in quantities not above approximately 2 to 3 wt.-%. In particular, α-sulfo fatty acid alkyl esters are preferred which have an alkyl chain with not more than 4 C atoms in the ester group, for example methyl ester, ethyl ester, propyl ester and butyl ester. Particularly advantageously, the methyl esters of the α-sulfo fatty acids (MES), but also the saponified disalts thereof, are used. Further suitable anionic surfactants are sulfonic fatty acid glycerol esters which represent mono, di and triesters as well as mixtures thereof, as are obtained during the production by esterification by a monoglycerol with from about 1 to about 3 mol fatty acid or during esterification of triglycerides with from about 0.3 to about 2 mol glycerol. The alkali and in particular sodium salts of sulfuric acid semiesters of C₁₂-C₁₈ fatty acid alcohols, for example of coconut oil alcohol, tallow fat alcohol, lauryl, myristyl, cetyl or stearyl alcohol or of C₁₀-C₂₀ oxoalcohols and those semiesters of secondary alcohols of these chain lengths are preferred as alk(en)yl sulfates. Further preferred are alk(en)yl sulfates of the named chain length, which contain a synthetic, straight-chained alkyl residue produced on a petrochemical basis, which possess degradation behavior analogous to the adequate compounds on the basis of fatty-chemical raw materials. Of interest from a washing-related technical aspect, C₁₂-C₁₆ alkyl sulfates and C₁₂-C₁₅alkyl sulfates as well as C₁₄-C₁₅ alkyl sulfates are in particular preferred. Also, 2,3-alkyl sulfates, which can be obtained as commercial products from Shell Oil Company under the name DAN®, are suitable anionic surfactants. Also suitable are the sulfuric acid monoesters of straight-chained or branched C₇-C₂₁ alcohols, such as 2-methyl-branched C₉-C₁₁ alcohols with on average 3.5 mol ethylene oxide (EO) or C₁₂-C₁₈ fatty acid alcohols with 1 to 4 EO, which C₇-C₂₁ alcohols are ethoxylated with 1 to 6 mol ethylene oxide. The preferred anionic surfactants also include the salts of alkyl sulfosuccinic acid, which are also called sulfosuccinates or sulfosuccinic acid esters, and which represent monoesters and/or diesters of sulfosuccinic acid with alcohols, preferably fatty alcohols and in particular ethoxylated fatty alcohols. Preferred sulfosuccinates contain C₈ to C₁₈ fatty alcohol residues or mixtures thereof. In particular, preferred sulfosuccinates contain a fatty alcohol residue which is derived from ethoxylated fatty alcohols which, considered per se, represent non-ionic surfactants. In turn, sulfosuccinates, the fatty alcohol residues of which are derived from ethoxylated fatty alcohols with concentrated homolog distribution, are particularly preferred. It is also likewise possible to use alk(en)yl sulfosuccinic acid with preferably 8 to 18 carbon atoms in the alk(en)yl chain, or the salts thereof. Fatty acid derivatives of amino acids, for example of N-methyltaurine (taurides) and/or of N-methylglycerol (sarcosides) come into consideration as further anionic surfactants. Particularly preferred are the sarcosides or the sarcosinates and here, above all, sarcosinates of higher and optionally singly or multiply unsaturated fatty acids such as oleyl sarcosinate. In particular, soaps come into consideration as further anionic surfactants. In particular, saturated fatty acid soaps, such as the salts of lauric acid, myristic acid, palmitic acid, stearic acid, hydrogenated erucic acid and behenic acid are in particular from natural fatty acids, for example coconut, palm kernel or tallow fatty acids or derived soap mixtures, are suitable. The known alkenyl succinic acid salts can also be used together with these soaps, or as a replacement for soaps.

The anionic surfactants, including the soaps in the form of the sodium, potassium or ammonium salts thereof, as well as soluble salts of organic bases, such as mono-, di- or triethanol amine, are present. Preferably, the anionic surfactants are present in the form of the sodium or potassium salts thereof, in particular in the form of sodium salts. Surfactants are contained in detergents in quantity details normally of from about 1 wt.-% to about 50 wt.-%, in particular of from about 5 wt.-% to about 30 wt.-%.

A detergent contains preferably at least one water-soluble and/or water-insoluble, organic and/or inorganic builder. The water-soluble organic builder substances include polycarboxylic acids, in particular citric acid and saccharic acids, monomeric and polymeric amino polycarboxylic acids, in particular glycine diacetic acid, methylglycine diacetic acid, nitrilotriacetic acid, iminodisuccinates such as ethylene diamine-N,N′-disuccinic acid and hydroxyimino disuccinates, ethylene diamine tetraacetic acid as well as polyaspartic acid, polyphosphonic acids, in particular aminotris(methylene phosphonic acid), ethylene diamine tetrakis(methylene phosphonic acid), lysine tetra(methylene phosphonic acid) and 1-hydroxyethane-1,1-diphosphonic acid, polymeric hydroxy compounds such as dextrin as well as polymeric (poly)carboxylic acids, polycarboxylates accessible in particular by oxidation of polysaccharides, polymeric acrylic acids, methacrylic acids, maleic acids and mixed polymer of same, which may also contain small proportions of polymerizable substances without carboxylic acid functionality polymerized therein. The relative average molecular mass of the homopolymers of unsaturated carboxylic acids is generally between about 5,000 g/mol and about 200,000 g/mol, that of the copolymers between about 2,000 g/mol and about 200,000 g/mol, preferably from about 50,000 g/mol to about 120,000 g/mol, in each case relative to the free salts thereof. A particularly preferred acrylic acid-maleic acid copolymer has a relative average molecular mass of from about 50,000 to about 100,000. Suitable, if also less preferred, compounds of this class are copolymers of acrylic acid or methacrylic acid with vinyl ethers, such as vinylmethyl ether, vinyl ester, ethylene, propylene and styrene, in which the proportion of acid is at least about 50 wt.-%. There can also be used as water-soluble organic builders terpolymers which contain two unsaturated acids and/or the salts thereof as monomers, as well as vinyl alcohol and/or a vinyl alcohol derivative or a carbohydrate as third monomer. The first acid monomer or the salt thereof is derived from a monoethylenically unsaturated C₃-C₈ carboxylic acid and preferably from a C₃-C₄ monocarboxylic acid, in particular from (meth)acrylic acid. The second acid monomer or the salt thereof can be a derivative of a C₄-C₈ dicarboxylic acid, wherein maleic acid is particularly preferred. In this case, the third monomeric unit is formed by vinyl alcohol and/or preferably an esterified vinyl alcohol. In particular, vinyl alcohol derivatives are preferred which represent an ester of short-chained carboxylic acids, for example C₁-C₄ carboxylic acids, with vinyl alcohol. Preferred polymers contain from about 60 wt.-% to about 95 wt.-%, in particular from about 70 wt.-% to about 90 wt.-% (meth)acrylic acid or (meth)acrylate, particularly preferably acrylic acid or acrylate, and maleic acid or maleinate as well as from about 5 wt.-% to about 40 wt.-%, preferably from about 10 wt.-% to about 30 wt.-% vinyl alcohol and/or vinyl acetate. Quite particularly preferred are polymers in which the weight ratio of (meth)acrylic acid or (meth)acrylate to maleic acid or maleinate is between about 1:1 and about 4:1, preferably between about 2:1 and about 3:1 and in particular between about 2:1 and about 2.5:1. Both the quantities and also the weight ratios are relative to the acids. The second acid monomer or the salt thereof can also be a derivative of an allyl sulfonic acid which is substituted in 2 position with an alkyl residue, preferably with a C₁-C₄ alkyl residue, or an aromatic residue which is derived preferably from benzene or benzene derivatives. Preferred terpolymers contain from about 40 wt.-% to about 60 wt.-%, in particular from about 45 to about 55 wt.-% (meth)acrylic acid or (meth)acrylate, particularly preferably acrylic acid or acrylate, from about 10 wt.-% to about 30 wt.-%, preferably from about 15 wt.-% to about 25 wt.-% methallyl sulfonic acid or methallyl sulfonate and as third monomer from about 15 wt.-% to about 40 wt.-%, preferably from about 20 wt.-% to about 40 wt.-%, of a hydrocarbon. This hydrocarbon can for example be a mono-, di-, oligo- or polysaccharide, wherein mono-, di- or oligosaccharides are preferred. Saccharose is particularly preferred. Supposedly weakened points are built into the polymer by using the third monomer, which points are responsible for the good biodegradability of the polymer. These terpolymers generally have a relative average molecular mass between about 1,000 g/mol and about 200,000 g/mol, preferably between about 200 g/mol and about 50,000 g/mol. Further preferred copolymers are those which have acrolein and acrylic acid/acrylic acid salt or vinyl acetate as monomer. The organic builders may be used in the form of aqueous solutions, preferably in the form of from about 30 to about 50 weight percent aqueous solutions, in particular for producing liquid agents. All named acids are generally used in the form of their water-soluble salts, in particular their alkali salts.

Such organic builders can if desired be contained in quantities of up to 40 wt.-%, in particular up to 25 wt.-% and preferably of from about 1 wt.-% to about 8 wt.-%. Quantities close to the named upper limit are preferably used in pasty or liquid, in particular water-containing, agents.

Water-soluble inorganic builder materials in particular polyphosphates, preferably sodium triphosphate, come into consideration. As water-insoluble inorganic builder materials, in particular crystalline or amorphous, water-dispersible alkali aluminosilicates are used, in quantities of not more than 25 wt.-%, preferably of from about 3 wt.-% to about 20 wt.-% and in particular in quantities of from about 5 wt.-% to about 15 wt.-%. The crystalline sodium aluminosilicates in detergent quality, in particular zeolite A, zeolite P and zeolite MAP and optionally zeolite X, are preferred among these. Quantities close to the named upper limit are preferably used in solid, particulate agents. Suitable aluminosilicates in particular do not have any particles with a particle size of more than about 30 μm and consist preferably up to at least about 80 wt.-% of particles with a size of less than about 10 μm. The calcium binding capability is generally in the range of from about 100 to about 200 mg CaO per gram.

Additionally, or alternatively to the named water-insoluble aluminosilicate and alkalicarbonate, further water-soluble inorganic builder materials can be contained. These include, in addition to the polyphosphonates such as sodium triphosphate, in particular the water-soluble crystalline and/or amorphous alkalisilicate builder. Such water-soluble inorganic builder materials are contained in the agents preferably in quantities of from about 1 wt.-% to about 20 wt.-%, in particular of from about 5 wt.-% to about 15 wt.-%. The alkalsilicates which can be used as builder materials preferably have a molar ratio of alkali oxide to SiO₂ of less than about 0.95, in particular of from about 1:1.1 to about 1:12, and may be present in amorphous or crystalline form. Preferred alkalisilicates are sodium silicates, in particular the amorphous sodium silicates, with a molar ratio Na₂O: SiO₂ of from about 1:2 to about 1:2.8. As crystalline silicates, which may be present alone or in a mixture with amorphous silicates, preferably crystalline sheet silicates of the general formula Na₂SiₓO₂ₓ₊₁.H₂O are used, in which x, the so-called modulus, is a number of from about 1.9 to about 4 and y is a number of from 0 to about 20 and preferred values for x are 2, 3 or 4. Preferred crystalline sheet silicates are those in which x takes on the values 2 or 3 in the named general formula. In particular, both β- and also δ-sodium disilicates are preferred (Na₂Si₂O₅.y H₂O). Also, practically water-free crystalline alkali silicates made from amorphous alkali silicates of the above-named general formula, in which x is a number of from about 1.9 to about 2.1, can be used in the agents. In a further preferred embodiment, a crystalline sodium sheet silicate with a modulus of from about 2 to about 3 is used, as can be produced from sand and soda. Sodium silicates with a modulus in the range of from about 1.9 to about 3.5 are used in a further embodiment. In a preferred embodiment of such agents, a granular compound of alkali silicate and alkali carbonate is used, as is commercially available for example under the name Nabion® 15.

As bleaching agents, there are taken into consideration those based on chlorine, such as in particular alkaline hypochlorite, dichloroisocyanuric acid and the salts thereof, but in particular also those based on peroxygen. In particular, organic peracids or peracid salts of organic acids, such as phthalimido-percaproic acid, perbenzoic acid, monoperoxyphthalic acid and diperdodecanoic acid and the salts thereof come into consideration as suitable peroxygen compounds, such as magnesium monoperoxyphthalate, hydrogen peroxide and, under the use conditions, hydrogen peroxide emitting inorganic salts such as perborate, percarbonate and/or persilicate, as do hydrogen peroxide inclusion compounds such as H₂O₂ urea adducts. Hydrogen peroxide can also be produced using an enzymatic system, i.e. an oxidase and the substrate thereof. Where solid peroxygen compounds are intended to be used, these can be used in the form of powders or granulates which can also be encapsulated in known manner. Particularly preferably, alkalipercarbonate, alkaliperborate-monohydrate or hydrogen peroxide in the form of aqueous solutions, which contain from about 3 wt.-% to about 10 wt.-% hydrogen peroxide, are used. If a detergent contains peroxygen compounds, these are present in quantities of preferably up to about 25 wt.-%, in particular of from about 1 wt.-% to about 20 wt.-% and particularly preferably of from about 7 wt.-% to about 20 wt.-%.

In particular, compounds which under perhydrolysis conditions produce optionally substituted perbenzoic acid and/or aliphatic peroxocarboxylic acids with 1 to 12 C atoms, in particular 2 to 4 C atoms, alone or in mixtures, are used as a bleach-activating compound which provides peroxycarboxylic acid under perhydrolysis conditions. Bleach activators which bear the O- and/or N-acyl groups in particular of the named C atom number and/or optionally substituted benzoyl groups are particularly preferred. Multiply acylated alkylene diamines, in particular tetraacetylethylenediamine (TAED), acylated glycolurils, in particular tetraacetyl glycoluril (TAGU), acylated triazine derivatives, in particular 1,5-diacetyl-2,4-dioxohexahydro-1,3,5-triazine (DADHT), N-acylimides, in particular N-nonanoylsuccinimide (NOSI), acylated phenolsulfonates or carboxylates or the sulfonic or carboxylic acids of same, in particular nonanoyl or isononanoyl or lauroyloxybenzol sulfonate (NOBS or iso-NOBS or LOBS) or decanoyl oxybenzoate (DOBA), the formal carbonic acid derivatives thereof such as 4-(2-decanoyloxy ethoxycarbonyloxy)-benzolsulfonate (DECOBS), acylated polyvalent alcohols, in particular triacetin, ethylene glycol diacetate and 2,5-diacetoxy-2,5-dihydrofurane as well as acetylated sorbitol and mannitol and the mixtures thereof (SORMAN), acylated sugar derivatives, in particular pentaacetylglucose (PAG), pentaacetylfructose, tetraacetylxylose and octaacetyllactose, acetylated, optionally N-alkylated glucamin and gluconolactone, and/or N-acylated lactam, for example N-benzoyl caprolactam, are preferred.

In addition to the compounds which form peroxocarboxylic acids under perhydrolysis conditions, further bleach-activating compounds, such as for example nitriles, from which perimide acids form under perhydrolysis conditions, may be present. These include in particular aminoacetonitrile derivatives with quaternized hydrogen atom according to the formula

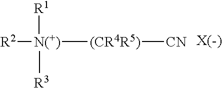

in which R¹ stands for —H, —CH₃, a C₂₋₂₄ alkyl or alkenyl residue, a substituted C₁₋₂₄ alkyl or C₂₋₂₄ alkenyl residue with at least one substituent from the group —Cl, —Br, —OH, —NH₂, —CN and —N(+)-CH₂—CN, an alkyl or alkenyl aryl residue with a C₁₋₂₄ alkyl group, or for a substituted alkyl or alkenyl aryl residue with at least one, preferably two, optionally substituted C₁₋₂₄ alkyl group(s) and optionally further substituents on the aromatic ring, R² and R³, independently of one another, are selected from —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂CH₂—O)ₙH with n=1, 2, 3, 4, 5 or 6, R⁴ and R⁵, independently of one another, have a meaning as indicated before for R¹, R² or R³, wherein at least 2 of the named residues, in particular R² and R³, also including the nitrogen atom and optionally further heteroatoms can be connected to one another in ring-closing manner, and then preferably form a morpholino ring, and X is a charge-equalizing anion, preferably selected from benzenesulfonate, toluenesulfonate, cumol sulfonate, the C₉₋₁₅ alkylbenzol sulfonates, the C₁₋₂₀ alkylsulfates, the C₈₋₂₂ carboxylic acid methylester sulfonates, sulfate, hydrogensulfate and the mixtures thereof, can be used. Also, sulfonimines and/or acylhydrazones transferring oxygen can be used.

Also, the presence of bleach-catalyzing transition metal complexes is possible. These are preferably selected from cobalt, iron, copper, titanium, vanadium, manganese and ruthenium complexes. Both inorganic and also organic compounds come into consideration as ligands in such transition metal complexes, those compounds including, in addition to carboxylates, in particular compounds with primary, secondary and/or tertiary amine and/or alcohol functions, such as pyridine, pyridazine, pyrimidine, pyrazine, imidazole, pyrazole, triazole, 2,2′-bispyridylamine, tris-(2-pyridylmethyl)amine, 1,4,7-triazacyclononane, 1,4,7-trimethyl-1,4,7-triazacyclononane, 1,5,9-trimethyl-1,5,9-triazacyclododecane, (Bis-41-methylimidazole-2-yl)-methyl))-(2-pyridylmethyl)-amine, N,N′-(Bis-(1-methylimidazole-2-yl)-methyl)-ethylenediamine, N-Bis-(2-benzimidazolylmethyl)-amino-ethanol, 2,6-Bis-(bis-(2-benzimidazolylmethyl)aminomethyl)-4-methylphenol, N,N,N′,N′-tetrakis-(2-benzimidazolylmethyl)-2-hydroxy-1,3-diaminopropane, 2,6-Bis-(bis-(2-pyridylmethyl)aminomethyl)-4-methylphenol, 1,3-Bis-(bis-(2-benzimidazolylmethy)aminomethyl)-benzene, sorbitol, mannitol, erythritol, adonitol, inositol, lactose, and optionally substituted saline, porphine and porphyrine. The inorganic neutral ligands include in particular ammonia and water. If not all coordination centers of the transition metal central atom are possessed by neutral ligands, the complex contains further, preferably anionic and among these in particular mono- or bidentate ligands. These include in particular the halides such as fluoride, chloride, bromide and iodide, and the (NO₂)⁻ group, i.e. a nitro ligand or a nitrito ligand. The (NO₂)⁻ group can also be bonded to a transition metal in chelating manner, or it can bridge two transition metal atoms asymmetrically or in η¹-O manner. Apart from the named ligands, the transition metal complexes can bear further, generally simply constructed ligands, in particular monovalent or polyvalent anion ligands. For example, nitrate, acetate, trifluoroacetate, formiate, carbonate, oxalate, perchlorate and complex anions such as hexafluorophosphate come into consideration. The anion ligands are intended to ensure the charge equalization between transition metal central atom and the ligand system. Also, the presence of oxo ligands, peroxo ligands and imino ligands is possible. In particular, such ligands can also be bridging, with the result that polynuclear complexes are produced. In the case of bridged, binuclear complexes, the two metal atoms in the complex do not have to be the same. Also, the use of binuclear complexes in which the two transition metal central atoms have different oxidation numbers is possible. If anion ligands are missing, or the presence of anion ligands does not lead to charge equalization in the complex, in the transition metal complex compounds to be used as contemplated herein, anionic counterions are present which neutralize the cationic transition metal complex. These anionic counterions include in particular nitrate, hydroxide, hexafluorophosphate, sulfate, chlorate, perchlorate, the halides such as chloride or the anions of carboxylic acids such as formiate, acetate, oxalate, benzoate or citrate. Examples of transition metal complex compounds which can be used are Mn(IV)₂(μ-O)₃(1,4,7-trimethyl-1,4,7-triazacyclononane)-di-hexafluorophosphate, [N,N′-Bis[(2-hydroxy-5-vinylphenyl)-methylene]-1,2-diaminocyclohexane]-manganese-(III)-chloride, [N,N′-Bis[(2-hydroxy-5-nitrophenyl)-methylene]-1,2-diaminocyclohexane]-manganese-(III)-acetate, [N,N′-Bis[(2-hydroxyphenyl)-methylene]-1,2-phenylenediamine]-manganese-(III)-acetate, [N,N′-Bis[(2-hydroxyphenyl)-methylene]-1,2-diaminocyclohexane]-manganese-(III)-chloride, [N,N′-Bis[(2-hydroxyphenyl)-methylene]-1,2-diaminoethane]-manganese-(III)-chloride, [N,N′-Bis[(2-hydroxy-5-sulfonatophenyl)-methylene]-1,2-diaminoethane]-manganese-(III)-chloride, manganese-oxalate complexes, nitropentaammine-cobalt(III) chloride, nitritopentaammine-cobalt(III)-chloride, hexaammine cobalt(III)-chloride, chloropentaammmine-cobalt(III)-chloride and peroxo-complex [(NH₃)₅Co—O—O—Co(NH₃)₅]Cl₄.

As enzymes which can be used in the agents, in addition to the laccases as contemplated herein, there come into consideration those from the class of amylases, proteases, lipases, cutinases, pullulanases, hemicellulases, cellulases, oxidases and peroxidases and mixtures thereof. Also, the use of one or more further laccases or multi-copper oxidases is possible as contemplated herein, in addition to the laccases as contemplated herein. Enzymatic active ingredients obtained from fungi or bacteria, such as Bacillus subtilis, Bacillus licheniformis, Bacillus lentus, Streptomyces griseus, Humicola lanuginosa, Humicola insolens, Pseudomonas pseudoalcaligenes, Pseudomonas cepacia or Coprinus cinereus, are particularly suitable. The enzymes can be adsorbed on supports and/or embedded in solid materials in order to protect them from premature inactivation. They are contained in the detergents or cleaning agents as contemplated herein, preferably in quantities of up to about 5 wt.-%, in particular of from about 0.2 wt.-% to about 4 wt.-%. If the agent as contemplated herein contains protease, it preferably has a proteolytic activity in the range of from approximately 100 PE/g to approximately 10,000 PE/g, in particular from about 300 PE/g to about 8,000 PE/g. If several enzymes are intended to be used in the agent as contemplated herein, this can be carried out by being incorporating the two or more enzymes which are separate or produced separately, in known manner, or by two or more enzymes produced together in a granulate.

In addition to water, the organic solvents which can be used in the detergents, in particular if present in liquid or pasty form, include alcohols with 1 to 4 C atoms, in particular methanol, ethanol, isopropanol and tert.-butanol, diols with 2 to 4 C atoms, in particular ethylene glycol and propylene glycol, as well as the mixtures thereof and the ethers which can be derived from the named classes of compounds. Such water-miscible solvents are present in the agents as contemplated herein, preferably in quantities of not more than about 30 wt.-%, in particular of from about 6 wt.-% to about 20 wt.-%.

To set a desired pH which does not result by itself by mixing the remaining components, the agents as contemplated herein can contain system-compatible and environmentally-compatible acids, in particular citric acid, acetic acid, tartaric acid, malic acid, lactic acid, glycolic acid, succinic acid, glutaric acid and/or adipic acid, but also mineral acids, in particular sulfuric acid, or bases, in particular ammonium or alkali hydroxides. Such pH regulators are contained in the agents as contemplated herein in quantities of preferably not more than about 20 wt.-%, in particular of from about 1.2 wt.-% to about 17 wt.-%.

Graying inhibitors have the task of keeping the dirt released from the textile fibers suspended in the liquor. For this, water-soluble colloids of mostly organic nature are suitable, for example starch, sizing material, gelatins, salts of ethercarboxylic acids or ethersulfonic acids of the starch or of the cellulose or salts of acid sulfuric acid esters of the cellulose or of the starch. Also, polyamides containing water-soluble acid groups are suitable for this purpose. Furthermore, ones other than the above-named starch derivatives can be used, for example aldehyde starches. Preferably, cellulose ethers are used, such as carboxymethyl cellulose (Na salt), methyl cellulose, hydroxyalkyl cellulose and mixed ethers such as methylhydroxyethyl cellulose, methylhydroxypropyl cellulose, methylcarboxymethyl cellulose and the mixtures thereof, for example in quantities of from about 0.1 to about 5 wt.-%, relative to the agents.

For example, detergents can contain derivatives of the diaminostilbene disulfonic acid or the alkali metal salts thereof as optical brighteners, although they are preferably free from optical brighteners for use as color detergents. For example, salts of 4,4′-Bis(2-anilino-4-morpholino-1,3,5-triazinyl-6-amino)stilbene-2,2′-disulfonic acid or similarly structured compounds, which instead of the morpholino group carry a diethanol amino group, a methylamino group, an anilino group or a 2-methoxyethylamino group, are suitable. Furthermore, brighteners of the type of substituted diphenyl styryls can be present, for example the alkali salts of 4,4′-Bis(2-sulfostyryl)-diphenyl, 4,4′-Bis(4-chloro-3-sulfostyryl)-diphenyl, or 4-(4-chlorostyryl)-4′-(2-sulfostyryl)-diphenyl. Also, mixtures of the above-named optical brighteners can be used.

In particular, when used in machine methods, it can be advantageous to add customary foam inhibitors to the agents. For example, soaps of natural or synthetic origin which have a high proportion of C₁₈-C₂₄ fatty acids are suitable as foam inhibitors. Suitable non-surfactant foam inhibitors are for example organopolysiloxanes and the mixtures thereof with microfine, optionally silanized silicic acid and paraffins, waxes, microcrystalline wax and mixtures thereof with silanized silicic acid or bis fatty acid alkylene diamides. Also, mixtures of different foam inhibitors are used with advantages, for example those made from silicones, paraffins or waxes. Preferably, the foam inhibitors, in particular silicone and/or paraffin-containing foam inhibitors, are bonded to a granular carrier substance which is soluble in water or dispersible. In particular, mixtures of paraffins and bistearylethylene diamide are preferred.

The production of solid agent does not present any difficulties and can take place in known manner, for example by spray-drying or granulation, wherein enzymes and possible further thermally sensitive ingredients such as for example bleaching agent are optionally added later. To produce agents with an increased bulk density, in particular in the range of from about 650 g/l to about 950 g/1, a method comprising an extrusion step is preferred.

To produce agents in tablet form, which are monophase or multiphase, monochromatic or polychromatic and in particular consist of one layer or of several, in particular of two layers, it is preferably assumed that all components—optionally per layer—are mixed together in a mixer and the mixture compressed by employing conventional tablet presses, for example eccentric presses or rotary presses, with pressing forces in the range of from approximately 50 to 100 kN, preferably at from about 60 to about 70 kN. In particular, in multilayer tablets it can be advantageous if at least one layer is precompressed. This is preferably carried out at pressing forces between about 5 and about 20 kN, in particular at from about 10 to about 15 kN. In this way, break-proof tablets which are sufficiently quickly soluble under application conditions are then obtained without problems, which tablets have breaking strength and bending strengths of normally from about 100 to about 200 N, but preferably more than 150 N. Preferably a tablet produced in this way has a weight of from about 10 g to about 50 g, in particular of from about 15 g to about 40 g. The tablet can take on any spatial form and can be round, oval or square, wherein also intermediate forms are also possible. Corners and edges are advantageously rounded. Round tablets preferably have a diameter of from about 30 mm to about 40 mm. In particular, the size of square or prismatic-designed tablets, which are predominantly introduced via the dosing device of the washing machine, is dependent on the geometry and the volume of this dosing device. Preferred embodiments by way of example have a base surface of (from about 20 to about 30 mm)×(from about 34 to about 40 mm), in particular of 26×36 mm or 24×38 mm.

Liquid or pasty agents in the form of conventional solvent-containing solutions are generally produced by simple mixing of the ingredients, which can be placed in the substance or as solution in an automatic mixer.

Solid and/or liquid detergents as contemplated herein can e.g. also be packed in portion bags (preferably self-dissolving), portion pouches, in particular also in multi-chamber pouches. Within the meaning of the present disclosure, the term “liquid” also includes any solid dispersions in liquids. Liquid agents as contemplated herein can also be multiphase, the phases can e.g. be vertical, thus arranged on top of each other or horizontal, thus side by side.

### EXAMPLES

The following examples explain the present disclosure without however limiting it:

### Example 1

Determining the activity of the laccases with the amino acid sequences SEQ ID NO. 1, SEQ ID NO. 3, SEQ ID NO. 5 and SEQ ID NO. 7:

Malvidin assay: Enzyme activity of the laccase was determined photometrically. For this, the anthocyanin primulin (Malvidin-3-galactoside chloride, CAS Number: 30113-37-2) was used as laccase substrate.

The absorption was measured in a Synergy 2™ microplate reader (BioTek Instruments GmbH, Bad Friedrichshall, Germany).

The substrate solution consisted of buffer (0.1 M NaAc; 0.1 M potassium phosphate, pH 8) and 31 mmol Primulin (Sigma). 50 μL of the laccase sample was added to 100 μL substrate solution in a microtiter plate and the reduction of absorption measured over 30 minutes at 30° C. as mAbs/min. Comparison samples were produced with water instead of the laccase sample.

Conversion into units:

The enzyme activity was calculated using the formula:

Activity [Malvidin mU/mL]=(total volume in the assay in μL*increase per min)/(sample volume in the Assay in mL*16000 L/mol/cm*layer thickness in cm)

The layer thickness with 150 μL capacity in the microtiter plates was 0.425 cm. The negative increase (−Abs/min) was converted to positive.

Using this method, the activities of the laccases purified from the wild-type residues were determined which were used for example 2:

The enzyme sample of the Pleurotus pulmonarius-purified wild-type residue shows an activity of −21.4 mAbs/min and thus 9 Malvidin mU/mL.

The enzyme sample of the Pleurotus ostreatus var. Florida-purified wild-type residue shows an activity of −20.4 mAbs/min and thus 9 Malvidin mU/mL.

The enzyme sample of the Lentinula edodes-purified wild-type residue shows an activity of −4.5 mAbs/min and thus 2 Malvidin mU/mL.

The enzyme sample of the Trametes species (Tsp, DSMZ 11309)-purified wild-type residue shows an activity of −29.5 mAbs/min and thus 13 Malvidin mU/mL.

ABTS assay:

For the ABTS assay, 245 μL 50 mM Na acetate buffer pH 4.5 was presented in a MTP, plus 30 μL 5 mM ABTS solution, plus 10 μL 2 mM H₂O₂, plus 15 μL sample, in corresponding dilution. A kinematic measurement was made at 420 nm, 30° C. for 10 min. The activity of ABTS radicals released can be calculated in mol per minute and ml using the molar extinction coefficient for ABTS.

Sample volume: 0.015 mL, test volume: 0.3 mL, eABTS=0.0368 cm2*mol-1, at 405-420 nm

Activity [μmol*min−1*ml−1]=(((DE/minSample−DE/minBlank)*p*R²)/(eABTS*test volume))*(test volume/sample volume)*dilution factor

Using this method, laccases expressed in heterologous manner in trichoderma were determined which are used for example 4:

Laccase from Led 2359 ABTS U/mL

Laccase from Ppu 6152 ABTS U/mL

Laccase from Tsp 3142 ABTS U/mL

### Example 2

First mini washing tests took place respectively with one of the following purified wild-type residues:

a. Pleurotus pulmonarius, in which the laccase with the amino acid sequence SEQ ID NO. 1 is present;
b. Pleurotus ostreatus var. florida, in which the laccase with the amino acid sequence SEQ ID NO. 3 is present;
c. Lentinula edodes, in which the laccase with the amino acid sequence SEQ ID NO. 5 is present;
d. Trametes species (Tsp, DSMZ 11309), in which the laccase with the amino acid sequence SEQ ID NO. 7 is present.

The test conditions were as follows: 40° C., 16° dH water, 1 h, laccase concentration: 1 Malvidin mU/mL

For this test, standardized soiled textiles were used. The following impurities were used:

### Currant CS12 [CFT (Center For Testmaterials) B. V. Vlaardingen, Netherlands]

### Blueberry CS15 [CFT (Center For Testmaterials) B. V. Vlaardingen, Netherlands]

### Tea EMPA 167 [EMPA: Eidgenössische Material-und Prüfanstalt (EMPA) Testmaterialien

AG [Federal materials and testing agency, Testmaterials], St. Gallen, Switzerland]

The punched out woven fabric (diameter=10 mm) was presented in microtiter plates, the wash liquor was pre-tempered to 40° C. The end concentration of the washing matrix was 4.06 g/L in 16° dH water.

Then, liquor and enzyme were added to the impurity, followed by an incubation for 1 h at 40° C. and 600 rpm.
Subsequently, the impurity was rinsed several times with clear water, left to dry and the brightness determined with a Minolta color-measurement apparatus Cm700d (CIE L*a*b*, D65/10°/SCI).
The cleaning performance was shown on the brightness of the washed woven fabric. The brighter the woven fabric, the better the cleaning performance. What was measured was the L value=brightness, the higher, the brighter.
Washing took place with a liquid detergent which contained no enzymes, optical brighteners and dyes. The composition is indicated in example 3.
Washing test with laccase from Trametes species (Tsp, DSMZ 11309)

Sample 1: only detergent as benchmark

Sample 2: Detergent plus laccase from Trametes species (Tsp, DSMZ 11309), 1 mU/mL

Sample 3: Detergent plus laccase from Pleurotus ostreatus (Sigma), 1 mU/mL

Result (sample 1 vs. sample 2):

| Impurity | Sample 1 | Sample 2 |
|---|---|---|
| Currant | 74.8 | 79.7 |
| Blueberry | 73.3 | 74.0 |
| Tea | 73.8 | 75.1 |

It can be seen clearly that the laccase shows a good performance on both anthocyanin-containing impurities (currant and blueberry). The tea stain has not become darker as is known with other laccases, but advantageously has clearly brightened. A significant improvement in performance can already be mentioned from 1 unit; indeed, here an improvement of 4.9 units has been achieved.

A commercially available laccase from Pleurotus ostreatus can be used on the same activity level as the laccase in sample 2, as negative control.

Result (sample 1 vs. sample 3):

| Impurity | Sample 1 | Sample 3 |
|---|---|---|
| Currant | 76.8 | 77.7 |
| Blueberry | 76.4 | 75.7 |
| Tea | 75.1 | 74.8 |

The detergent power from sample 3 has not recognizably improved over sample 1.

Washing test with laccase from Pleurotus ostreatus var. Florida (Pos)

Sample 1: only detergent as benchmark

Sample 2: Detergent plus Laccase from Pos, 1 mU/mL

Sample 3: Detergent plus laccase from Pleurotus ostreatus (Sigma), 1 mU/mL

Result (sample 1 vs. sample 2):

| Impurity | Sample 1 | Sample 2 |
|---|---|---|
| Currant | 74.8 | 78.1 |
| Blueberry | 73.3 | 75.7 |
| Tea | 73.8 | 74.1 |

It can be seen clearly that the laccase shows a good performance on both anthocyanin-containing impurities (currant and blueberry) and no darkening on tea, as is brought about by other laccases. A significant improvement in performance can already be mentioned from 1 unit; here indeed 3.3 and respectively 2.4 units of improvement have been achieved.

For the negative control see a).

Washing test with laccase from Lentinula edodes

Sample 1: only detergent as benchmark

Sample 2: Detergent plus laccase from Lentinula edodes (Led), 1 mU/mL

Sample 3: Detergent plus laccase from Pleurotus ostreatus (Sigma), 1 mU/mL

Result (sample 1 vs. sample 2):

| Impurity | Sample 1 | Sample 2 |
|---|---|---|
| Currant | 74.8 | 76.5 |
| Blueberry | 73.3 | 76.9 |
| Tea | 73.8 | 77.0 |

It can be seen clearly that the laccase shows a good performance on both anthocyanin-containing impurities (currant and blueberry). The tea stain has not only not become darker as is known with other laccases, but advantageously has clearly brightened. A significant improvement in performance can already be mentioned from 1 unit; here indeed 4.7 and respectively 3.4 units of improvement have been achieved.

For the negative control see a).

Washing test with laccase from Pleurotus pulmonarius

Sample 1: only detergent as benchmark

Sample 2: Detergent plus laccase from Pleurotus pulmonarius (Ppu), 1 mU/mL

Sample 3: Detergent plus laccase from Pleurotus ostreatus (Sigma), 1 mU/mL

Result (sample 1 vs. sample 2):

| Impurity | Sample 1 | Sample 2 |
|---|---|---|
| Currant | 74.8 | 77.4 |
| Blueberry | 73.3 | 76.6 |
| Tea | 73.8 | 74.3 |

It can be seen clearly that the laccase shows a good performance on both anthocyanin-containing impurities (currant and blueberry). The tea stain has not only not become darker as is known with other laccases, but advantageously has clearly brightened. A significant improvement in performance can already be mentioned from 1 unit; here indeed 2.6 and respectively 3.3 units of improvement have been achieved.

For the negative control see a).

### Example 3

Detergent formulation:

This formulation was used in example 2. The dose was 69 g/17 L:

| | % active substance | % active substance |
|---|---|---|
| Chemical name: | (raw material) | in formulation |
| Demin. water | 100 | Residue |
| Alkylbenzene sulfonic | 96 | 4.40 |
| acid | | |
| Anionic surfactant | 70 | 5.60 |
| C12-C18 fatty acid Na | 100 | 2.40 |
| sodium | | |
| Nonionic surfactant | 100 | 4.40 |
| Phosphonate | 32 | 0.20 |
| Citric acid | 100 | 1.43 |
| NaOH | 50 | 0.95 |
| Antifoam | 10 | 0.01 |
| Glycerol | 99.5 | 2.00 |
| Preservative | 100 | 0.08 |
| Ethanol | 93 | 1.00 |

Without opt. brightener, perfume, dye and enzymes.

### Example 4

Washing test in the launder-O-meter:

The test conditions were as follows:
40° C., 16° dH water, 1 h, 200 mL washing liquor, 10 steel balls, 16.8 g impurities and connective tissue
Laccase concentration: 25 ABTS U/mL

For this test, mainly natural impurities were used, produced from juices and marmalades. The following impurities were used:

Wild huckleberry jam, Darbo
Basel black cherry marmalade, Mövenpick
Huckleberry juice, Rabenhorst
Cranberry juice, Rabenhorst
Preiselbeer juice, Rabenhorst
Holunderbeer juice, Rabenhorst

### Blueberry CS15, [CFT (Center For Testmaterials) B. V. Vlaardingen, Netherlands]

The impurities are applied on cotton nonwoven fabric (approx. 5 cm diameter), brushed in and stored for at least 1 week.

After the launder-o-meter has run for 1 h at 40° C., the impurities are rinsed 4× with water, then spun and air-dried. The brightness is measured with a Minolta CR400-1 color-measuring apparatus after being wrung. The color value Y is determined, compared with the blank (detergent without enzymes).

For the washing test, the following 3 laccases were used which have been expressed in heterologous manner in trichoderma:

Laccase from Pleurotus pulmonarius (Ppu), with SEQ ID NO. 1
Laccase from Lentinula edodes (Led), with SEQ ID NO. 5
Laccase from Trametes species (Tsp) (Tsp, DSMZ 11309), with SEQ ID NO. 7.

Result:

The values represent the delta-Y for the detergent Blank without enzymes. A significant improvement is considered to be only 1 unit of change.

| | Laccase | Laccase | Laccase |
|---|---|---|---|
| | from Led | from Ppu | from Tsp |
| Wild huckleberry jam | 1.5 | 2.2 | 2.4 |
| Basel black cherry marmalade | 1.8 | 1.5 | 2.0 |
| Huckleberry juice | 0.4 | 1.7 | 0.4 |
| Cranberry juice | 0.6 | 1.1 | 1.0 |
| Lingonberry juice | 0.8 | 1.5 | 0.4 |
| Holunderbeer juice | 1.0 | 1.1 | 1.0 |
| CS-15 blueberry | 1.3 | 1.8 | 3.0 |

A significant cleaning performance can be seen on stains containing anthocyanin. The laccase from Pleurotus pulmonarius shows the broadest performance spectrum.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A laccase, comprising an amino acid sequence which is at least about 70% identical to the amino acid sequence indicated in SEQ ID NO. 1, over the entire length thereof.

2. The laccase according to claim 1, wherein the laccase is coded by a nucleic acid sequence which is at least about 70% identical to the nucleic acid sequence indicated in SEQ ID NO. 2, over the entire length thereof.

3. A detergent, comprising at least one laccase according to claim 1.

4. The detergent according to claim 3, wherein the concentration of the at least one laccase is in an amount of from about 0.001 to about 0.15 wt.-% relative to active protein.

5. The detergent according to claim 3, wherein the detergent is utilized in the temperature range of from about 5° C. to about 95° C.

6. The detergent according to claim 3, wherein the detergent further comprises additional mediators which are selected from 2,2,6,6-tetramethyl-1-piperidinyloxy, 1-hydroxy-benzotriazol, 2,2′-azinobis-3-ethylbenzthiazol-6-sulfonate, N-Hydroxy-acetanilide, 2,5-xylidin, ethanol, copper, 4-methylcatechol, N-hydroxyphthalimide, gallic acid, tannic acid, quercetin, syringic acid, guaiacol, dimethoxybenzyl alcohol, phenol, violuric acid, phenol red, bromophenol blue, cellulose, p-coumaric acid, rooibos, o-cresol, dichloroindophenol, hydroxybenzotriazol, cycloheximide, vanillin, or combinations thereof.

7. The laccase according to claim 1, wherein the laccase is utilized for improving the detergent power of a detergent.

8. A method for washing textiles in aqueous solutions which comprise surfactant, wherein the method comprises utilizing a solution which comprises surfactant and at least one laccase according to claim 1.

9. The laccase according to claim 1, wherein the amino acid sequence is at least about 80% identical to the amino acid sequence indicated in SEQ ID NO. 1, over the entire length thereof.

10. The laccase according to claim 9, wherein the amino acid sequence is at least about 90% identical to the amino acid sequence indicated in SEQ ID NO. 1, over the entire length thereof.

11. The laccase according to claim 10, wherein the amino acid sequence is at least about 95% identical to the amino acid sequence indicated in SEQ ID NO. 1, over the entire length thereof.

12. The laccase according to claim 2, wherein the nucleic acid sequence is at least about 80% identical to the nucleic acid sequence indicated in SEQ ID NO. 2, over the entire length thereof.

13. The laccase according to claim 12, wherein the nucleic acid sequence is at least about 90% identical to the nucleic acid sequence indicated in SEQ ID NO. 2, over the entire length thereof.

14. The laccase according to claim 13, wherein the nucleic acid sequence is at least about 95% identical to the nucleic acid sequence indicated in SEQ ID NO. 2, over the entire length thereof.

15. The detergent according to claim 4, wherein the concentration of the at least one laccase is in an amount of from about 0.005 to about 0.06 wt.-% relative to active protein.

16. The detergent according to claim 5, wherein the detergent is utilized in the temperature range of from about 20° C. to about 60° C.

17. The detergent according to claim 16, wherein the detergent is utilized in the temperature range of from about 30° C. to about 40° C.

18. The laccase according to claim 7, wherein the laccase is utilized for improving the detergent power of a detergent when removing colored stains and impurities.

19. The laccase according to claim 7, wherein the laccase is utilized for improving the detergent power of a detergent when removing impurities containing anthocyanin.


SEQUENCE LISTING (8 sequences)

SEQ ID NO: 1 - protein, 533 aa, Pleurotus pulmonarius
MAVAFIALVSLALALVRVEASIGPRGTLNIANKVISPDGFSRSTVLAGGSYPGPLIKGKTGDRFHINVVNELADTSMPIDTSIHWHGLFAKGHNWADGPAMVTQCPIVPGHSFLYEFDVPDQAGTFWYHSHLGTQYCDGLRGPLVIYSKNDPHKRLYDVDDESTVLTVGDWYHAPSLSLTGVPHPDSTLFNGLGRSLNGPASPLYVMNVVKGKRYRIRLINTSCDSNYQFSIDGHTFTVIEADGENTQPLQVDQVQIFAGQRYSLVLNANQAVGNYWIRANPNSGDPGFANQMNSAILRYKGAHSIDPTTPEQNATNPLREYNLRPLVKKPAPGKPFPGGADHNINLNFAFDPATALFTANNFTFVPPTVPVLLQILSGTRDAHDLAPAGSIYDIKLGEVVEITMPALVFAGPHPLHLHGHTFAVVRSAGSSTYNYENPVRRDVVSIGDDPTDNVTIRFVADNAGPWFLHCHIDWHLDLGFAVVFAEGVNQTAVANPVPEAWNNLCPLYNSSNPSKLLMGTNAIGRLPAPLKA

SEQ ID NO: 2 - DNA, 1602 bp, Pleurotus pulmonarius
atggcggttgcattcattgcgcttgtttcacttgccttagcactcgtacgcgtcgaggccagcatcgggccccgcggaacgctgaacatagcgaacaaagtcatcagcccagatggattttctcgctcaaccgtgctcgccggtggctcctacccgggcccgttgatcaaggggaagaccggtgataggtttcatatcaatgtcgtgaacgagctcgccgatacgtcgatgccaatcgacaccagtatccactggcatggtctcttcgccaagggccacaattgggcagatggccctgccatggttacgcaatgcccaatcgttccgggccactcgtttttgtacgaattcgacgtccctgatcaagctggaacattttggtatcactcccatctggggacacagtactgtgatggactgcgagggccattggtcatctactcgaagaacgacccccacaagcgtttgtacgatgtcgacgatgaatccacggtgctgaccgttggtgactggtaccacgccccctcattatcacttactggagtgccccatcccgactcaacactcttcaatggcctcggacgttccctcaacggtccagcatcgccgctatacgttatgaacgtagtcaaaggcaaacgctatcgtattcggctcatcaacacctcctgcgactccaactaccaattttccattgacggacataccttcactgttatcgaagctgatggggagaacacacagcctctgcaggtcgatcaagtgcaaatttttgcaggccagcgctattcgcttgtcctcaacgcgaatcaggcggtcggcaactactggatccgcgcaaaccccaacagcggcgatcccggtttcgcgaaccagatgaactctgccatcctccggtacaaaggcgcacacagcatcgaccccacgacccccgagcagaacgctaccaaccccctccgcgaatacaacctccgcccgctcgtcaagaagcccgcgccaggcaaaccattccccggaggcgccgaccacaacatcaacctaaacttcgctttcgatcctgccaccgctctgttcactgcgaacaatttcacgtttgtcccccctactgttccagtattgttgcagatcttgtcgggcacacgcgatgcgcatgatttggcccctgctgggtcgatttatgacattaagctaggagaagtcgttgagatcactatgcctgcacttgtatttgctggaccacatccccttcatttacatgggcataccttcgctgtagttcgcagtgccggcagcagcacatataactacgagaatcccgtccgtagagatgttgtatccatcggtgatgacccaacggacaacgtcaccatccgattcgtagcagacaacgcaggtccttggttcctgcactgccacatcgactggcatcttgacctcggcttcgcggtcgtctttgccgaaggagtaaatcagaccgcagtagccaaccccgtacctgaagcctggaacaacctctgccccctatacaacagctctaacccatctaaacttctaatgggcactaatgccattggccgtctgcctgcgccactgaaggcatga

SEQ ID NO: 3 - protein, 521 aa, Pleurotus ostreatus var. florida
MVLSTKLAALVASLPFVLAATKKLDFHIRNDVVSPDGFERRAITVNGIFPGTPVILQKNDKVQINTINELTDPGMRRSTSIHWHGLFQHKTSGMDGPSFVNQCPIPPNSTFLYDFDTAGQTGNYWYHSHLSTQYCDGLRGSFIVYDPNDPLKHLYDVDDESTIITLADWYHDLAPHAQNQFFQTGSVPIPDTGLINGVGRFKGGPLVPYAVINVEQGKRYRFRLIQISCRPFFTFSIDNHTFDAIEFDGIEHDPTPAQNIDIYAAQRASIIVHANQTIDNYWIRAPLTGGNPAGNPNLDISLIRAILRYKGAPAVEPTTVATTGGHKLNDAEMHPIAQEGPGNLGTGPPDMAITLNIAQPNPPFFDINGISYLSPSVPVLLQMLSGARKPQDFLPSEQVIILPANKLIEVSIPGAGAHPFHLHGHTFDIVRTSNSDVVNLVNPPRRDVLPINGGNTTFRFFSGNSGAWFLHCHIDWHLEAGLAVVFAERPAEVNEGEQAQIVTQDWRTLCPAYDGLAPEFQ

SEQ ID NO: 4 - DNA, 1566 bp, Pleurotus ostreatus var. florida
atggtgctctctactaagctcgctgctcttgtggcttcgctccccttcgttcttgctgcgacgaagaaacttgacttccacattcgaaacgacgttgtctcaccagacggcttcgagcgcagggcgatcacagtcaatggcatcttccctgggacgcccgttatactgcagaagaacgacaaggtccagatcaatactatcaatgaactgacggaccctggcatgcgtcgcagtacctccatccattggcacggtttattccaacataaaacctccggcatggacgggccttctttcgttaaccagtgcccgattccccccaactctactttcctttacgacttcgataccgcaggacagactggaaactactggtaccactcccacttgtctacgcaatattgcgacggtcttcgtggttctttcatcgtttatgatcccaatgatcctctgaagcacctgtacgatgttgatgatgagagcaccatcatcaccctggcagattggtatcatgaccttgctccccacgctcaaaaccagttcttccaaactggttcggtcccgattcccgacactggcctgatcaacggtgttggacgattcaagggcggtcctcttgtcccttacgccgtcatcaacgttgagcagggcaagcgatacagattccgcctcatccagatttcatgtcgtcccttcttcaccttctctattgacaaccacacattcgatgcaattgaattcgacggcatagagcacgacccgacacccgcccagaacattgatatctatgctgctcaacgtgcatccatcatcgtccacgccaaccagaccatcgacaactactggatccgtgcgccactcacagggggaaaccccgcaggtaaccctaacctggacatatcgttgatcagggccatccttcgttacaagggtgctcccgctgtcgagcccaccacagttgcgactactggaggccacaagctcaatgatgccgagatgcacccaattgctcaggaaggtcccggaaacttgggcaccggccccccagacatggccatcaccttgaacatcgcccagccgaaccctccattcttcgatatcaacggcatttcgtatctctctccttcggttccagtgttgctccagatgcttagcggtgctcgcaaaccacaagacttcctgccatcggaacaagtcatcatcttgcccgccaacaagctcattgaagtctctatacccggtgccggcgcgcatcctttccatttgcacggtcataccttcgacattgtccgcacttcgaatagcgacgtcgtgaaccttgtcaacccaccgcgtcgcgacgtattgcctatcaacggtggaaacacaacattccgtttcttctctggcaactccggtgcatggttcctccattgccatattgactggcatcttgaagctggtctcgctgttgtcttcgcggaacgacctgctgaagtgaacgagggcgaacaggctcagatcgtcacccaagactggaggacgctttgcccagcttatgatggcctcgctcccgagttccagtaa

SEQ ID NO: 5 - protein, 515 aa, Lentinula edodes
MGFQSFTSLLSLVALVPSAFAVLETTGDLVISNAAVSPDGFSRTAVLAGGGVVGELVTGNKGDNFQINVVNELTDDTMLRSTTVHWHGIFQEGTNWADGPAFINQCPIAANNSFLYDFTVSDQAGTFWYHSHLATQYCDGLRGPMVIYDPDDPYADLYDVDDDTTIISLMDWYHAKAETLTFPAPDATLINGLGRYSGGNATDLAVITVTSGTRYRFRLINLACDPNFVFSIDNHTMTVIEVDGVNHEQLVVDSIQIFAAQRYSFILEANQSVDNYWIRADPSTGTAGYTGGINSAILRYSGAADSEPGTPAVTSVLALNETQLVPLENPGAPGEPEVGGVDYALNLAMAFTGTAFTINGYSFTSPTVPVLLQILSGAETADSLLPTGSVYSLPTNSTIEISIPSGGLAGFPHPFHLHGHTFDVVRSAGSTTYNYVNPVRRDVVSTGAAGDNVTIRFTTDNAGPWFLHCHIDWHLEAGLAIVFAEDVADWNTTQTPSTAWDDLCPIYDALSADQL

SEQ ID NO: 6 - DNA, 1548 bp, Lentinula edodes
atgggctttcaatcttttacctccctcctttcactggtcgcacttgttccgtcagcttttgctgtcctggaaacaactggtgacctcgtcatttccaacgctgcagtcagccccgatggtttcagccgtaccgctgtccttgctggcggtggggtcgtaggtgaacttgtcacaggaaacaagggcgataacttccaaatcaatgttgtgaacgagttgacggacgataccatgttgcgatctacaaccgtccattggcacggcatcttccaagagggaaccaattgggctgatggacctgcattcatcaatcagtgtcccatagcagcaaacaactccttcttatacgacttcaccgtttctgatcaagcagggacattttggtatcacagccatctcgcgacgcaatattgcgatggtcttcgaggtcctatggtcatctatgatcctgatgatccttatgcggacttgtacgatgttgacgacgacaccaccattatcagtctcatggattggtatcacgcaaaggctgaaactctcaccttcccggctccggatgctaccttaatcaacggtcttggacgttactctggaggtaatgccactgatttggctgtcatcacagtgacttcagggactcgataccgattccgcttaattaatttggcttgtgatcccaacttcgtgttcagcattgataatcatacgatgactgtcatcgaagtagatggagtcaaccacgagcaattagtcgttgattctatccagattttcgctgcccaacgatactcattcattcttgaagctaatcaatccgtggacaattattggattcgcgcggacccctcaaccggtaccgctggatatacaggagggatcaattctgccattctgcgatacagtggagccgccgacagtgagcctggaactccggcagttacaagtgttctggctctaaacgaaactcagcttgtgcctttggaaaaccctggtgctccaggagaacctgaagtaggaggcgtagattatgctttaaatctggctatggcatttactggaacggctttcactatcaatggctatagctttacttcaccgacggtacccgtcttgcttcaaatcctcagcggtgcggaaacggccgattcgcttttgcctacggggtctgtttactccttgcccaccaactcgaccatcgagatttctattccttctggaggacttgccggtttccctcaccccttccatcttcacggtcataccttcgatgttgtgcgcagtgctggtagtacgacctataactacgtcaacccggtccgtcgagatgtcgtcagcactggagccgccggggacaatgtaacaatccgtttcactacagataacgcgggtccttggttccttcactgccacattgactggcatttggaggccggtcttgccattgtctttgctgaagatgttgccgattggaacactacccagactccctccactgcttgggatgatctgtgccccatctacgacgctctttctgctgaccaactttga

SEQ ID NO: 7 - protein, 520 aa, Trametes species (Tsp, DSMZ 11309)
MSRFQSLLAFVVASLAAVAHAAIGPTADLTISNAEVSPDGFARQAVVVNNVTPGPLVAGNKGDRFQLNVIDNLTNHTMLKSTSIHWHGFFQKGTNWADGPAFVNQCPISSGHSFLYDFQVPDQAGTFWYHSHLSTQYCDGLRGPFVVYDPNDPHASLYDVDNDDTVITLADWYHTAAKLGPAFPLGADATLINGLGRSPSTTAADLAVINVTKGKRYRFRLVSLSCDPNHTFSIDGHDLTIIEVDSINSQPLVVDSIQIFAAQRYSFVLNADQDVGNYWIRANPNFGNVGFAGGINSAILRYDGADPVEPTTTQTTPTKPLNEVDLHPLATMAVPGSPVAGGVDTAINMAFNFNGTNFFINGASFVPPTVPVLLQIISGAQNAQDLLPSGSVYSLPANADIEISFPATAAAPGAPHPFHLHGHAFAVVRSAGSTVYNYDNPIFRDVVSTGTPAAGDNVTIRFRTDNPGPWFLHCHIDFHLEAGFAVVFAEDIPDVASANPVPQAWSDLCPIYDALDVNDQ

SEQ ID NO: 8 - DNA, 1563 bp, Trametes species (Tsp, DSMZ 11309)
atgtcgaggtttcagtctctgctcgccttcgtcgtcgcctccctcgcggctgtggcccatgccgccattgggcccaccgctgacctcaccatctccaatgccgaggtcagccccgatgggttcgctcgtcaggctgtggttgtcaacaatgttaccccaggacccctcgtcgcgggcaacaagggtgaccgcttccaactcaatgtcatcgacaacctcacgaaccacactatgctgaagagcacgagtattcactggcatggcttcttccagaaggggacaaactgggctgatggtcccgcgtttgtgaaccagtgccctatttcctctgggcactcgttcctctacgatttccaggttcccgaccaggccggtaccttctggtaccacagccacttgtccactcagtactgtgacggcctgcggggtcctttcgttgtgtacgatcccaatgacccgcacgcgagcttgtatgacgtcgacaatgacgacaccgtgatcaccctcgcggattggtaccacactgccgcgaagctcggccccgcgttcccactgggcgcggatgctacccttatcaacgggctcgggcgctcccccagcaccacggcggcggacctcgcggtcatcaacgtcacgaagggtaaacgctaccgtttccgcctggtttccctgtcgtgtgaccccaaccacacgttcagcatcgatggtcatgacttgacgatcatcgaggtggactccatcaattcccaacctctggtggttgactccatccagattttcgctgcgcagcggtactcctttgtgttgaatgccgatcaggacgtcgggaactactggattcgcgccaaccccaactttggcaacgtcggatttgcggggggtatcaactcggccatcctgcgctatgacggcgccgacccggttgagcccaccacgactcagactacgccgaccaagcccctgaacgaggtcgacttgcacccgctcgccaccatggctgtgcccggctccccggtcgcaggtggtgttgacacggctatcaacatggccttcaactttaacggtaccaactttttcatcaacggcgcgagctttgtgccccccaccgtgccggtcctgctccagatcatcagcggcgcccaaaacgcccaggatctcctcccgtctggcagcgtctactccctcccggcgaacgcggacatcgagatctcgttccctgctaccgcggctgctccgggtgcccctcaccccttccacttgcacggtcacgccttcgccgtcgttcgcagcgctggcagcaccgtctacaactacgacaaccccatcttccgcgacgtcgtcagcacggggacgcctgcggccggtgacaacgtcaccatccgcttccgcaccgacaaccccggtccgtggttcctccactgccacatcgacttccacctcgaggccggcttcgcggtcgtgttcgcggaggacatccccgacgtcgcgtcggcgaaccctgtccctcaggcgtggtctgatctgtgccccatctacgacgcgctcgatgtcaacgaccagtag